(19) United States
(12) Patent Application Publication
(10) Pub. No.: US 2024/0018181 A1
(43) Pub. Date: Jan. 18, 2024

(54) PROCESS FOR THE PRODUCTION OF 21-(ACETYLOXY)-17-(PROPIONYLOXY)-PREGN-4-ENE-3,20-DIONE

(71) Applicant: Industriale Chimica S.R.L., Milano (IT)

(72) Inventors: Roberto Lenna, S. Giorgio Su Legnano (IT); Claudio Delfrate, Rho (IT); Davide Rigamonti, Erba (IT); Silvia Genovese, Novara (IT)

(21) Appl. No.: 18/031,902

(22) PCT Filed: Feb. 5, 2021
(86) PCT No.: PCT/IB2021/050953

§ 371 Date: Apr. 14, 2023

(30) Foreign Application Priority Data
Oct. 19, 2020 (IT) 102020000024640
Dec. 28, 2020 (IT) 102020000032530

(51) Int. Cl. C07J 5/00

(52) U.S. Cl. CPC C07J 5/0053

(57) ABSTRACT

The present invention relates to a process for the preparation of 21-(acetyloxy)-17-(1-oxopropoxy)-pregn-4-ene-3,20-dione (VI) having the formula below: Compound (VI) can be used as a precursor for the synthesis of Clascoterone, a steroid used for the treatment of acne.

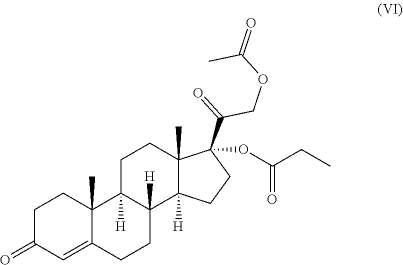

### FIELD OF THE INVENTION

The present invention relates to the field of processes for the synthesis of active ingredients for pharmaceutical use, and in particular to a process for the preparation, on an industrial scale, of 21-(acetyloxy)-17-(1-oxopropoxy)-pregn-4-ene-3,20-dione, a compound having the structural formula (VI) reported below:

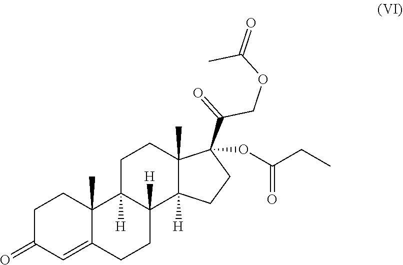

a useful precursor for the synthesis of 21-hydroxy-17-(1-oxopropoxy)-pregn-4-ene-3,20-dione, also known by the name of Clascoterone.

Clascoterone is a steroid with a pregnane skeleton that, appropriately formulated, has recently been approved by the United States Food and Drug Administration (FDA) to treat acne in pediatric patients, from 12 years of age, and adults. The structural formula of Clascoterone is shown below:

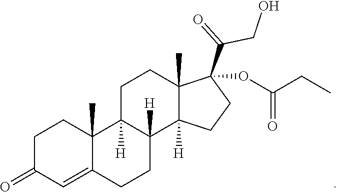

### STATE OF THE ART

Clascoterone is described in U.S. Pat. No. 3,152,154 dated 1964. As shown above, this compound is a 17α monoester of a 17α, 21-dihydroxy steroid.

According to the teachings of U.S. Pat. No. 3,152,154, 17-monoesters of 17α,21-dihydroxy steroids can be obtained by chemical hydrolysis in the presence of acid catalysis of corresponding 17α,21-(1′-alkoxy)1′-pregnanes (orthoesters) of the following type:

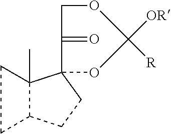

The experimental description reported in U.S. Pat. No. 3,152,154 does not provide any details relating to reaction yields and quality of the products obtained.

The orthoesters described in U.S. Pat. No. 3,152,154, in turn, can be prepared following the procedure described in U.S. Pat. No. 3,147,249. This second patent also does not provide any details regarding the reaction yields and quality of the products obtained.

Specifically, for the preparation of Clascoterone the starting compound for preparing the orthoester to be hydrolyzed would be 17,21-dihydroxy-pregn-4-ene-3,20-dione, a compound known by the name “Cortexolone”, having the formula structure shown below:

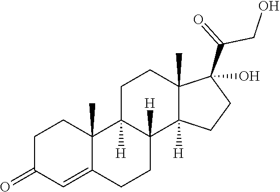

On the market, however, this compound is only available in laboratory amounts, not in the quantities needed for industrial production.

Another possible precursor of Clascoterone is the compound 17,21-bis(1-oxopropoxy)-pregn-4-ene-3,20-dione, compound of formula (VII) shown below:

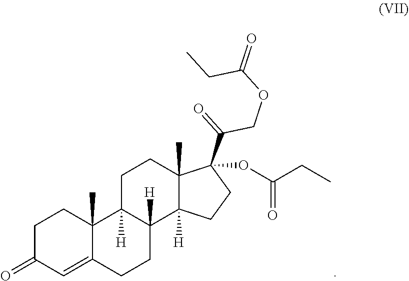

Compound (VII) can be prepared according to what described in patent application WO 2009/019138 A2, following the indications given in the article “Acylation of 17-hydroxy-20-ketosteroids”, R. B. Turner, J. Am. Chem. Soc. 1953, 75, 14, 3489-3492. The acid hydrolysis of compound (VII) requires, however, relatively long times and generates not negligible amounts of by-products.

WO 2009/019138 A2 also suggests the selective enzymatic hydrolysis of symmetrical diesters, i.e. wherein the radical R of the two ester groups is the same, using lipase according to the following reaction:

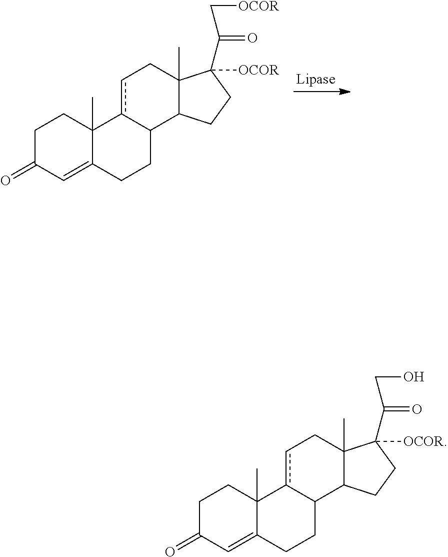

The object of the present invention is to provide a new intermediate useful for the synthesis of Clascoterone, as well as to provide a process that can be used at an industrial level for the synthesis of said intermediate.

### SUMMARY OF THE INVENTION

This object is achieved with the present invention that, in a first aspect thereof, relates to a process for the synthesis of 21-(acetyloxy)-17-(1-oxopropoxy)-pregn-4-ene-3,20-dione, compound of formula (VI):

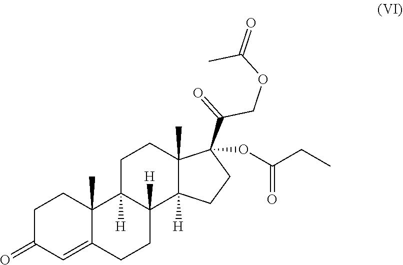

that can be used as a precursor in the synthesis of Clascoterone, said process comprising the following steps:
a) reaction of 17α-hydroxy-progesterone (I) with pyrrolidine to give compound (II), 17-hydroxy-3-(1-pyrrolidinyl)pregna-3,5-dien-20-one:

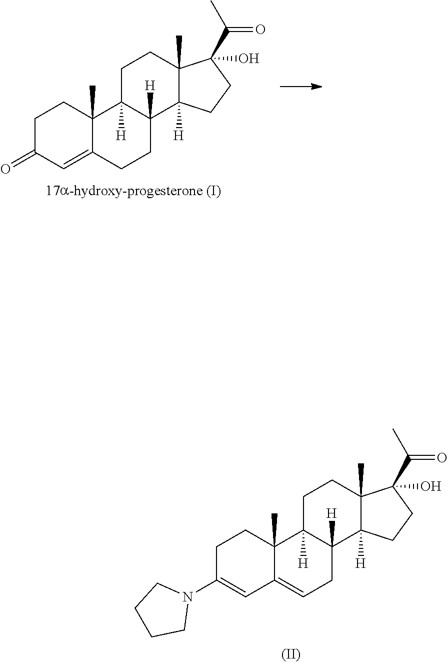

b) reaction of compound (II) first with hydrochloric acid then with bromine to give intermediate (III), a mixture of (21-chloro/21-bromo)-17α-hydroxy-3-(1-pyrrolidinium-1-yliden)-pregn-4-en-20-one chloride:

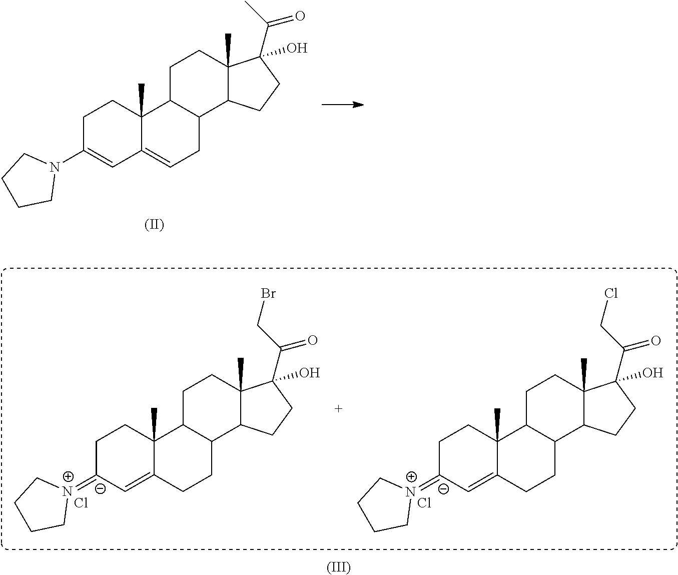

c) basic hydrolysis of intermediate (III) to obtain intermediate (IV), the corresponding mixture of 21-chloro/21-bromo-17α-hydroxypregn-4-en-3,20-dione:

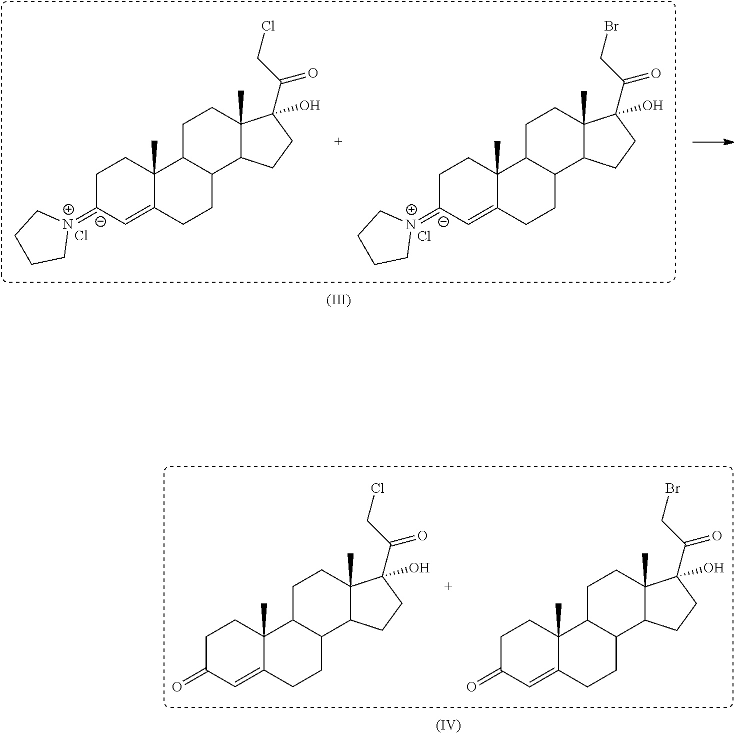

d) reaction of intermediate (IV) with acetic acid to obtain compound (V), 21-acetoxy-17α-hydroxypregn-4-en-3,20-dione:

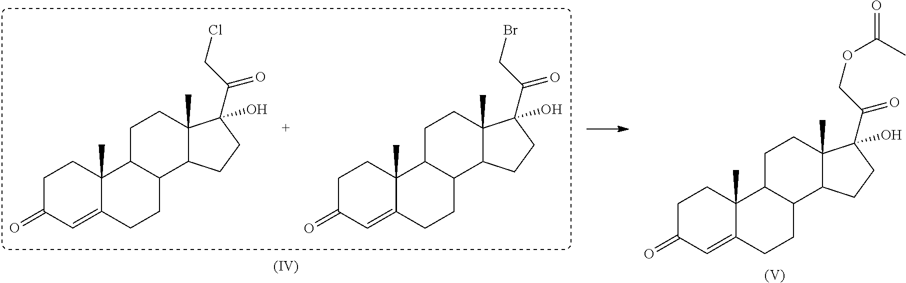

e) reaction of compound (V) with perchloric acid and propionic anhydride to obtain compound (VI), 21-(acetyloxy)-17-(1-oxopropoxy)-pregn-4-ene-3,20-dione:

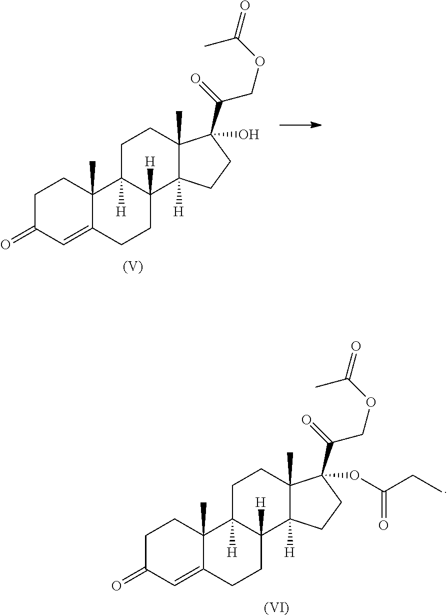

The process of the invention may further comprise an additional step f), of selective hydrolysis of compound (VI) to give Clascoterone:

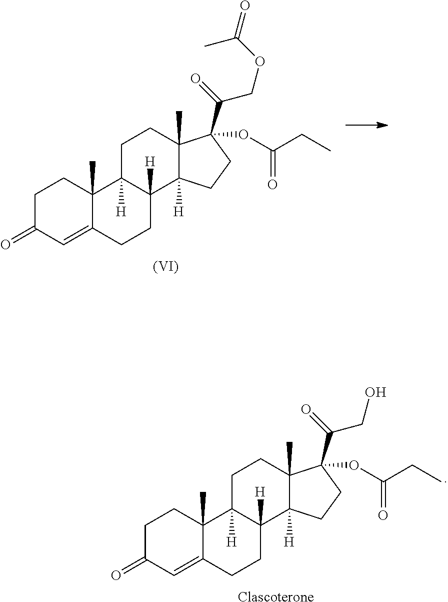

Step f) may be carried out by either chemical or enzymatic route.

In a second aspect thereof, the invention relates to compound (VI), 21-(acetyloxy)-17-(1-oxopropoxy)-pregn-4-ene-3,20-dione.

In a third aspect thereof, the invention relates to obtaining Clascoterone by enzymatic hydrolysis of compound (VI) operating with a flow reactor.

Finally, in a fourth aspect thereof, the invention relates to Clascoterone solvated with dimethyl sulfoxide.

### BRIEF DESCRIPTION OF THE FIGURES

FIG. 1 shows the HPLC chromatogram of compound 21-(acetyloxy)-17-(1-oxopropoxy)-pregn-4-ene-3,20-dione obtainable by the process of the invention.

FIG. 2 shows the XPRD diffraction spectrum of compound 21-(acetyloxy)-17-(1-oxopropoxy)-pregn-4-ene-3,20-dione obtainable by the process of the invention.

FIG. 3 shows the DSC thermogram of compound 21-(acetyloxy)-17-(1-oxopropoxy)-pregn-4-ene-3,20-dione obtainable by the process of the invention.

FIG. 4 shows the XPRD diffractogram of Clascoterone solvated with dimethyl sulfoxide and relative angle data and relative intensity of the peaks.

FIG. 5 shows the DSC thermogram of Clascoterone solvated with dimethyl sulfoxide.

FIG. 6 shows the FT-IR spectrum of Clascoterone solvated with dimethyl sulfoxide.

FIG. 7 shows the XPRD diffractogram of Clascoterone solvated with methanol.

### DETAILED DESCRIPTION OF THE INVENTION

The inventors found that the hydrolysis of a “non-symmetrical” diester provides better results than the hydrolysis of a symmetrical ester in the production of Clascoterone.

In the following description, when a ratio between an amount of a solvent and a compound is provided in terms of “volumes per weight”, the volume of the solvent is understood to be measured in milliliters and the weight of the compound in grams. Furthermore, for simplicity or clarity of representation, in some cases the stereochemical configuration of some atoms of the steroid skeleton is not shown in the figures herein; in these cases it is understood that the stereochemistry of the molecule corresponds to the natural configuration of steroids.

The term “non-symmetrical” diester means a structure of the following type:

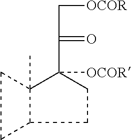

wherein the alkyl radicals R and R′ are different.

The non-symmetrical diester subjected to experimental check shows a more favorable behavior to acid hydrolysis than the symmetrical 17,21-bis(1-oxopropoxy)-pregn-4-ene-3,20-dione of formula (II) described in WO 2009/019138 A2; to avoid confusion with compound (II) of the present invention (the process intermediate 17-hydroxy-3-(1-pyrrolidinyl)pregna-3,5-dien-20-one), the compound (II) of WO 2009/019138 A2 will be denoted in the present description as compound (VII).

In fact, by carrying out acid hydrolysis reactions, in parallel and under the same conditions (perchloric acid in dichloromethane-methanol at 10-12° C.), on compound (VI) of the present invention and on compound (VII) of WO 2009/019138 A2, the inventors observed that the reaction with compound (VI) is completed in 37 hours (residual compound (VI) <3%) while 57 hours are required to reach the same result starting from compound (VII).

Furthermore, the composition of the mixture at the end of the reaction is also different and, as resulting from data in Table 1 below, the best result in terms of Clascoterone yield is obtained using compound (VI) (percentage concentrations shown in the table are calculated from areas of peaks in HPLC tests):

TABLE 1

| | | Products (%) | | | |
|---|---|---|---|---|---|
| | Reaction | | “Trans- | | Residual |
| Reagent | time (h) | Cortexolone | posed” | Clascoterone | reagent |
| (VI) | 37 h 20′ | 12.3 | 9.9 | 73.6 | 2.7 |
| (VII) | 57 h | 22.8 | 10.4 | 61.0 | 2.4 |

Except for residual unreacted reagent, the only by-product present in comparable amounts in the product of the two reactions is the one referred to as “transposed”, whose formation, as described in the article “Corticosteroid 17α-monoesters from 17α,21-cyclic orthoesters”, R. Gardi et al., Tetrahedron Letters (13) 1961, pages 448-451, cannot be suppressed as it is specific to the reaction product under the reaction conditions and does not depend on the starting substrate. The transposition reaction between positions 17 and 21 of the steroid is summarized below:

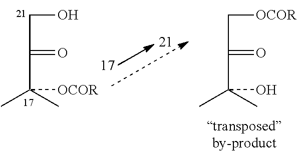

Monoesters in position 17 having a free hydroxyl group in position 21 are characterized by instability under acid reaction conditions that cause the migration of the acylating group from position 17 to position 21, according to the reaction mechanism outlined below:

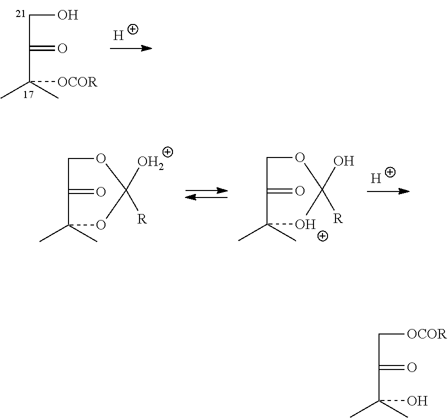

In the first aspect thereof, the invention relates to a process for the synthesis of 21-(acetyloxy)-17-(1-oxopropoxy)-pregn-4-ene-3,20-dione requiring the five synthetic steps a) to e) reported above.

Step a) consists in the reaction of compound (I) with pyrrolidine to give the corresponding enamine, compound 17-hydroxy-3-(1-pyrrolidinyl)pregna-3,5-dien-20-one (II), and it is carried out preparing a suspension of compound (I) in an alcohol, bringing this suspension to reflux, and then adding pyrrolidine. The starting compound (I), 17α-hydroxy-progesterone, is widely available on the market and does not require the synthesis of an orthoester as a process intermediate.

Pyrrolidine is used in a molar excess between 20 and 60%, preferably 40%, with respect to compound (I).

Alcohols that can be used to prepare the suspension are ethanol, isopropanol and, preferably, methanol.

The reaction mixture is kept at reflux for a time between 1 and 3 hours, preferably between 1.5 and 2.5 hours.

The obtained compound (II) is isolated by crystallization-precipitation from the reaction solvent.

Step b) consists in the reaction of enamine (II) first with hydrochloric acid then with bromine to give intermediate (III), a mixture of (21-chloro/21-bromo)-17α-hydroxy-3-(1-pyrrolidinium-1-yliden)-pregn-4-en-20-one chloride.

The reaction is carried out at a temperature between 10 and 40° C., preferably between 20 and 30° C.

The reaction solvent is an alcohol selected from methanol, isopropanol and, preferably, ethanol. The alcohol is used in an amount between 15 and 50 volumes, preferably between 15 and 30 volumes, with respect to the weight of compound (II).

Hydrochloric acid is used in the form of a 33% solution, by weight, in ethanol or isopropanol; the amount of this solution used in the reaction ranges from 1 to 3 times by weight with respect to the weight of compound (II), preferably 1.5 times.

The amount of bromine added, in moles, ranges between 1.0 and 3 times with respect to the moles of compound (II), preferably 1.5 times.

Bromine is added in the form of a solution in ethanol, in a bromine:ethanol volume ratio between 1:20 and 1:45, preferably 1:25. Before the addition to the solution prepared in the first part of this step, the bromine solution in ethanol is cooled down to a temperature between −50 and −60° C., preferably to −55° C. The addition of the bromine solution takes place over a time between 20 minutes and 2 hours, preferably between 80 and 100 minutes.

The intermediate (III) obtained at the end of step b) can be crystallized using a 1 to 4 carbon atoms linear or branched alcohol, an ether or a mixture thereof; the preferred solvent for crystallization of intermediate (III) is methyl tert-butyl ether (MTBE).

The reaction result is a mixture of (21-chloro/21-bromo)-17α-hydroxy-3-(1-pyrrolidinium-1-yliden)-pregn-4-en-20-one chloride, intermediate (III), which is used as it is in the continuation of the synthesis since both products react in the same way to give the desired 21-acetoxy product; for this reason, in this description, mixture (III) is denoted as a single reaction intermediate.

The minor component of mixture (III), 21-chloro-steroid, is present in a percentage ranging from 5 to 30%.

In an alternative embodiment, step b) could be carried out by directly reacting enamine (II) with hydrobromic acid, obtaining only the compound 21-bromine as intermediate (III) in this case.

Step c) of the process of the invention consists in the basic hydrolysis of intermediate (III) to obtain the corresponding 21-chloro/21-bromo-17α-hydroxypregn-4-en-3,20-dione mixture; this mixture too is used as such in the following process reaction, so that in the present description it is denoted as a single intermediate, intermediate (IV).

The reaction can be performed in an aqueous mixture of acetone, methanol or ethanol, wherein water is present in amounts of less than 50% by volume. A water/methanol mixture is preferably used, in which the volume of methanol is greater than 70% of total volume.

The base to be used may be selected from NaHCO₃, Na₂CO₃, KHCO₃ or K₂CO₃; KHCO₃ is preferably used in an amount in moles greater than 2 times with respect to the moles of intermediate (III).

The reaction temperature is of between 10° C. and the reflux temperature of the mixture; the reaction is preferably maintained at a temperature between 20 and 30° C.

The reaction time is between 2 and 16 hours, preferably between 4 and 6 hours.

The obtained intermediate (IV) can be crystallized using as solvents methyl-t-butyl ether (MTBE), ethyl acetate, acetonitrile, acetone, methyl ethyl ketone (MEK), methyl isobutyl ketone (MIBK), a 1 to 4 carbon atoms linear or branched alcohol, or mixtures thereof; preferably for this operation a 1:1 (v/v) MEK-MeOH mixture with the hot-cold technique is used. This technique, well known to those skilled in organic synthesis, consists in heating the product to be purified in the presence of a solvent. The resulting suspension and/or solution is then cooled. The solid product is filtered while the impurities present remain in solution.

In the next step, d), intermediate (IV) is reacted to give compound (V), 21-acetoxy-17α-hydroxypregn-4-en-3,20-dione.

The reaction can be carried out with glacial acetic acid in a solvent selected from dimethylformamide (DMF), acetone, methyl ethyl ketone (MEK), methyl isobutyl ketone (MIBK), methanol, ethanol, 2-propanol, toluene, or mixtures thereof, in the presence of an inorganic base selected from KHCO₃, NaHCO₃, K₂CO₃, Na₂CO₃ or an organic base selected from triethylamine (TEA), trimethylamine (TMA) or pyridine. Preferably operations are carried out in acetone or methyl ethyl ketone (MEK) with glacial acetic acid and triethylamine (TEA). Alternatively, the reaction can be carried out with sodium acetate or potassium acetate.

The reaction time is between 1 and 24 hours and the temperature is between 20° C. and the reflux temperature of the mixture; preferably operations are carried out for a time between 4 and 6 hours at the reflux temperature of the mixture.

The obtained compound (V) can be crystallized using solvents such as methyl-t-butyl ether (MTBE), acetone, methyl ethyl ketone (MEK), methyl isobutyl ketone (MIBK), a 1 to 4 carbon atoms, linear or branched alcohol or mixtures thereof; the preferred solvents are methyl ethyl ketone (MEK) and ethanol.

Finally, step e) of the process of the invention consists in the reaction of compound (V) with perchloric acid and propionic anhydride to obtain compound (VI), 21-(acetyloxy)-17-(1-oxopropoxy)-pregn-4-ene-3,20-dione.

The reaction is carried out by diluting compound (V) in dichloromethane (DCM), in an amount between 10 and 50 volumes, preferably 25 volumes of DCM with respect to the steroid weight, at a temperature between −25 and +25° C., preferably between −25 and −15° C. The reaction time can range from 5 to 60 minutes, preferably between 5 and 25 minutes.

Propionic anhydride is used in a molar ratio of between 6:1 and 9:1, preferably between 6:1 and 8:1 with respect to the steroid.

Compound (VI) can be purified by crystallization from ethyl acetate, isopropyl acetate, methyl ethyl ketone (MEK), methyl isobutyl ketone (MIBK), acetone, methanol, ethanol, 2-propanol, acetonitrile, toluene, THF, or methyl-THF.

In one embodiment thereof, the process of the invention includes a further step, f), consisting in the selective hydrolysis of compound (VI) to give Clascoterone.

Step f) can be carried out by acid hydrolysis, in similar conditions to those described in U.S. Pat. No. 3,152,154 for hydrolysis of orthoesters. For example, the reaction can be carried out under the conditions reported above for the comparison between the compounds (VI), of the invention, and (VII), of the known art, i.e. with perchloric acid in dichloromethane-methanol at 10-12° C.; as previously mentioned, under these conditions the acid hydrolysis of compound (VI) of the invention requires 37 hours to complete.

Hydrolysis of compound (VI) can also be carried out enzymatically, either by operating with a traditional batch reactor or by operating with a flow reactor.

For example, a sample of 21-(acetyloxy)-17-(1-oxopropoxy)-pregn-4-ene-3,20-dione (VI), reacted at 44-46° C. in toluene/n-butanol in a multi-necked flask equipped with mechanical stirrer and thermometer in the presence of LIPOMOD™ 34MDP Lipase (Biocatalysts, 115 U/mg), was hydrolyzed to 21-hydroxy-17-(1-oxopropoxy)-pregn-4-ene-3,20-dione (Clascoterone).

Similarly, but operating with an easy-Medchem E-series flow reactor from Vapourtec Ltd, Bury St Edmunds, (GB), equipped with a column packed with Novozym® 435 (Candida Antarctica Lipase B supported on acrylic resin; column sold by Strem Chemicals Inc., Bischheim, France), compound (VI) dissolved in toluene/n-butanol is hydrolyzed to 21-hydroxy-17-(1 -oxopropoxy)-pregn-4-ene-3,20-dione (Clascoterone).

In the enzymatic hydrolysis, the enzyme can be used in a free form in the reaction mixture but is preferably used in a supported form.

The reaction can be carried out under static conditions, but it is preferably carried out under flow conditions.

The reaction temperature is between 40 and 80° C., preferably between 50 and 70° C.

The solvent mixture to be used in the reaction is comprised of toluene and a linear alcohol, wherein the major component is toluene. Alcohols that can be used are methanol, ethanol, 1-propanol and, preferably, n-butanol.

The content of n-butanol in the toluene/n-butanol mixture is calculated with respect to the moles of compound (VI). From 1 to 10 moles, preferably between 2.5 and 5 moles, of n-butanol per mole of compound (VI) are used.

In one embodiment of the invention, Clascoterone can be obtained as a solvate from dimethyl sulfoxide (DMSO). In the solvate, Clascoterone and DMSO are present in a 1:1 molar ratio, as determined by NMR analysis. The solvate from DMSO can be obtained directly from the solution, after the enzymatic reaction, by replacing the reaction solvent with DMSO or through a solid intermediate consisting of a metastable solvate with methanol, as described in Example 11.

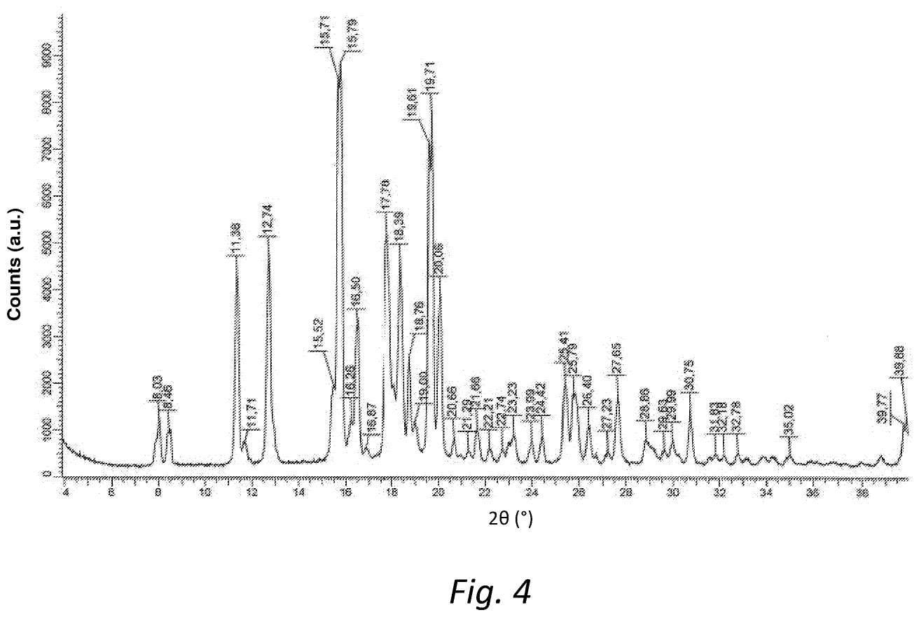

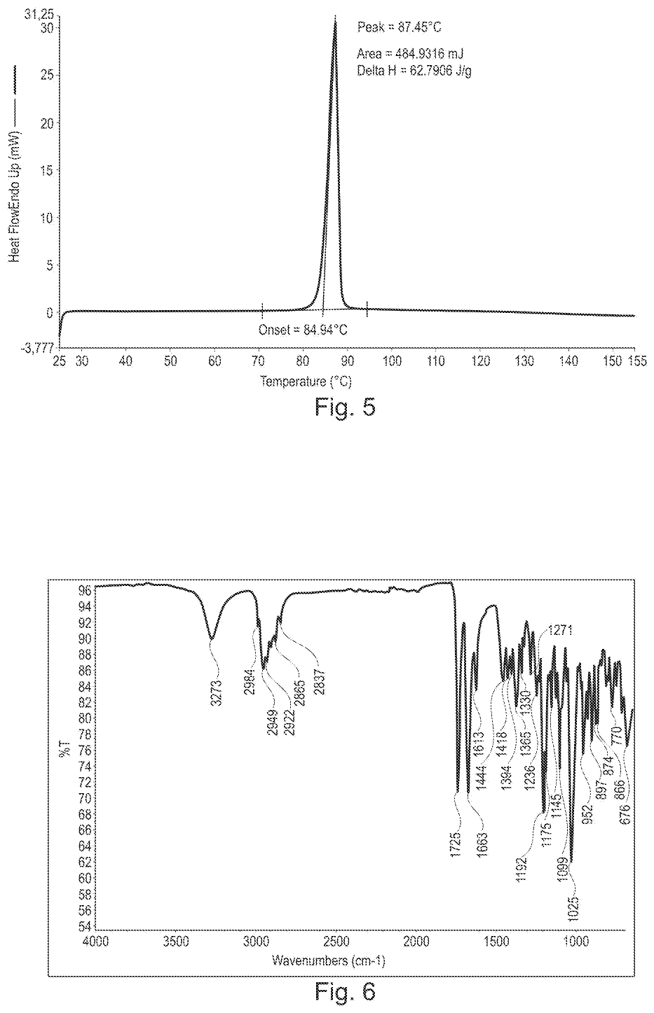

This solvate has a powder diffraction spectrum (XPRD) as shown in FIG. 4, a DSC thermogram as shown in FIG. 5, and an FT-IR spectrum as shown in FIG. 6.

The XPRD diffractogram is characterized by two intense double peaks, with reflections at 15.71° and 15.79° 2θ for the first double peak, and reflections at 19.61° and 19.71° 2θ for the second double peak; other characteristic peaks in the XPRD diffractogram of this solvate are at 11.38°, 12.74°, 16.50°, 17.78°, 18.39°, 18.76° and 20.06° 2θ; all these peak values are to be considered with an approximation of ±0.2°2θ.

The DSC thermogram, obtained in nitrogen and with a heating rate of 10° C./min, shows a single intense endothermic event with a peak at 87.45° C.

Once obtained, the solvate of Clascoterone with DMSO can be recrystallized several times from this solvent, until the desired level of purity is obtained. The process of recrystallization from a solvent is well known to those skilled in the art and consists in forming a solution of the substance to be purified in the desired solvent by bringing the system to a suitable temperature, about 65° C. in the case of DMSO, and then allowing it to cool until solidification of the compound, that can then be recovered using known methods such as filtration.

Clascoterone solvate from DMSO is particularly useful in pharmaceutical compositions for topical applications or where it is necessary to increase the permeability of a body tissue towards an active ingredient.

The use of compounds as active components of DMSO-based formulations is described, for example, in U.S. Pat. No. 3,711,602, dated 1973, wherein many of the examples refer to steroids.

The invention will be further illustrated by the following examples.

### INSTRUMENTS, METHODS AND EXPERIMENTAL CONDITIONS

NMR: JEOL 400 YH (400 MHz) NMR Spectrometer; JEOL Delta Software v5.1.1; spectra recorded in deuterated solvents such as: chloroform-d, D 99.8%, containing 0.1% (v/v) tetramethylsilane (TMS) as an internal standard; and chloroform-d, “100%”, D 99.96%, containing 0.03% (v/v) TMS, CD₃OD and DMSO-d₆.

TLC: MERCK: TLC Silica gel 60 F₂₅₄ Aluminium sheets 20×20 cm, cod. 1.0554.0001.

TLC Stain: Cerium phosphomolybdate: 25 g of phosphomolybdic acid and 10 g of cerium (IV) sulfate are dissolved in 600 ml of H₂O. 60 ml of 98% H₂SO₄ are added and the volume is brought to 1 L with H₂O. The plate is impregnated with the solution and then heated until the products are detected.

UPLC-MS: UPLC-MS Waters Acquity chromatographic system equipped with PDA and QDa detectors.

UPLC-MS Method:
Column: Waters Acquity BEH C18, 2.1(ID)×50 (L) mm, 7 μm;
Flow rate: 0.8 ml/min;
Mobile phase: acetonitrile/water from 1:1 (0-0.5′) to 9:1 (0.5′-2.5′);
pH modifier: 0.01% Formic acid;
UV detector: 244 nm;

XPRD: Bruker® D2 Phaser diffractometer (2^{nd} ed.) operating in Bragg-Brentano geometry and equipped with a 6-position rotating multisampler. The X-ray source is an X-ray tube with a copper anode, operated at 30 kV and 10 mA. The analytical wavelength used is copper Kα (λ=1.54184 Å). The Kβ radiation is filtered through a nickel filter. The X-ray detector is a linear solid-state detector, LYNXEYE model. The samples were layered as a thin layer on silicon sample holders of the “zero background” type. The diffractogram is recorded in the 4.0-40.0° 2θ angle range with increments of 0.016° and a scan rate of 1.0 s/increment in the case of the solvate from DMSO, while a scan rate of 0.25 s/increment was used in the case of the metastable solvate from methanol.

The data were analyzed using the DIFFRAC.EVA software (Bruker).

DSC: Diamond DSC instrument (Perkin Elmer) operated in a nitrogen atmosphere. The samples were prepared in 40 μL aluminum crucibles with lids and were closed using the appropriate press before analysis. The analysis was carried out at a constant heating rate equal to 10° C./min in the range of 25-210° C.

ATR-FTIR: FTIR Nicolet 6700 spectrophotometer (Thermo Fischer Scientific) equipped with an ATR Smart iTR (Thermo Fisher Scientific) module with diamond crystal. The acquisition was carried out by performing 64 scans in the range 4000-650 cm⁻¹, at a resolution of 4 cm⁻¹ for the measurement of both the analytical sample and the blank (measurement in the absence of a sample), which was acquired immediately before the measurement of the sample and automatically subtracted from it. The display of the spectrum and its analysis were performed using Omnic software (Thermo Fisher Scientific).

### NOTES

The water used in the experimental descriptions is intended to be pure water, unless otherwise stated.

Organic solvents used in the experimental descriptions are intended to be of a “technical” grade, unless otherwise stated.

Reagents and catalysts used in the experimental descriptions are intended to be of commercial quality, unless otherwise indicated.

### EXAMPLE 1

This example relates to step a) of the process of the invention, from 17α-hydroxy-progesterone (I) to 17-hydroxy-3-(1-pyrrolidinyl)pregna-3,5-dien-20-one (II):

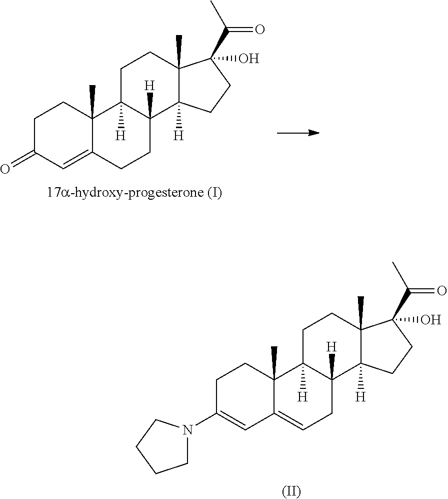

148.1 g of 17α-hydroxy-progesterone (I) are suspended in 740 ml of methanol. The suspension is heated to reflux (65° C.), without observing solubilization of the solid. 52.4 ml of pyrrolidine are added dropwise: complete dissolution of the starting product and almost immediate re-precipitation of enamine (II) are observed. The mixture is kept under stirring at this temperature for 2 hours. It is then cooled, first to room temperature and then to 0° C. for 1 h. It is filtered through Buchner, washing with 200 ml of cold methanol. The solid is dried under vacuum at 25° C. for 10 h, obtaining 166.2 g of compound (II) as an off-white solid.

17-α-Hydroxy-Progesterone (I) Analysis:

¹H-NMR, CDCl₃: 5.74 (1H, s, H-4); 2.77 (1H, s, OH-17); 2.72-2.65 (1H, m); 2.47-2.26 (4H, m); 2.29 (3H, s, H-21); 2.06-2.01 (1H, m); 1.90-1.81 (2H, m); 1.77-1.56 (7H, m); 1.46-1.33 (3H, m); 1.19 (3H, s, H-19); 1.17-1.07 (1H, m); 1.02-0.95 (1H, m); 0.77 (3H, s, H-18).

MS: 331 (M⁺+1).

Compound (II) Analysis:

¹H-NMR, CDCl₃: 5.07-5.06 (1H, m, H-6); 4.78 (1H, s, H-4); 3.15-3.12 (4H, m, N—CH₂); 2.74 (1H, br, OH); 2.71-2.65 (1H, m); 2.33-2.29 (2H, m); 2.28 (3H, s, H-21); 2.21-2.15 (1H, m); 1.91-1.56 (12H, m); 1.47-1.24 (4H, m); 1.11-1.05 (1H, m); 1.01 (3H, s, H-19); 0.76 (3H, s, H-18).

### EXAMPLE 2

This example relates to step b) of the process of the invention, from enamine (II) to intermediate (III), mixture (21-chloro/21-bromo)-17α-hydroxy-3-(1-pyrrolidinium-1-yliden)-pregn-4-en-20-one chloride:

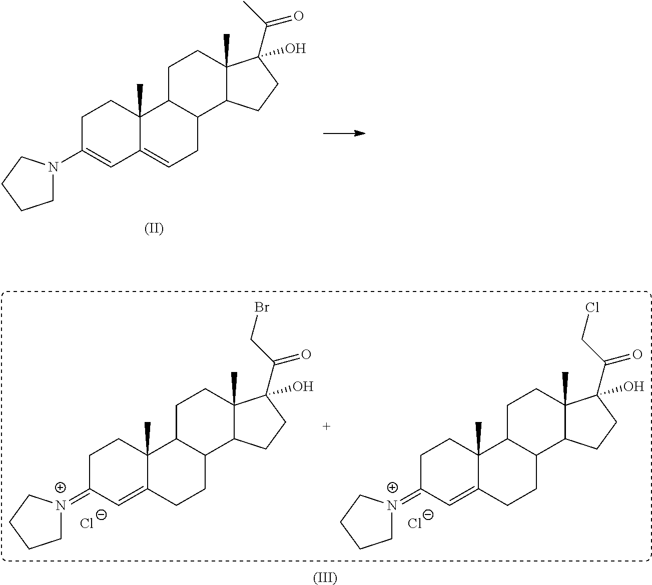

83 g of compound (II) obtained in the previous example are suspended in 1660 ml of ethanol at 20-25° C., under a nitrogen atmosphere. 125.6 g of HCl in ethanol (33% w/w) are added: complete dissolution is observed. A solution of bromine in ethanol (16.6 ml di bromine in 415 ml of ethanol), previously prepared and cooled to −55° C., is then added dropwise over about 90 minutes. Formation of a precipitate is observed towards the end of the addition. After the end of the addition, the mixture is kept under stirring for about 1 h at 20-25° C. and it is monitored by TLC: the starting material almost completely disappeared. The solvent is removed under vacuum at 45° C. by rotavapor, stripped 3 times with MTBE (450 ml each), the last time leaving a total volume of about 330 ml. It is cooled to 0° C. and kept under stirring for 1 hour. It is then filtered through Buchner, washing with cold MTBE. The product is dried for 2 h at 45° C. under vacuum, obtaining 106.6 g of intermediate (III) as a white powder.

Intermediate (III) Analysis:

¹H-NMR, DMSO: 6.51 (1H, s, H-4); 5.62 (1H, br, OH-17); 4.60 (1H, part A of an AB system, J_{AB}=15 Hz, H-21); 4.37 (1H, part B of an AB system, J_{AB}=15 Hz, H-21); 3.98-3.78 (4H, m, N—CH₂); 2.89-2.74 (2H, m); 2.61-2.55 (3H, m); 2.05-1.19 (16H, m); 1.12 (3H, s, H-19); 1.09-0.99 (1H, m); 0.95-0.89 (1H, m); 0.56 (3H, s, H-18).

In the spectrum there are also the following peaks belonging to the 21-chloro-derivative of the iminium: 5.59 (1H, br, OH-17); 4.79 (1H, part A of an AB system, J_{AB}=17 Hz, H-21); 4.48 (1H, part B of an AB system, J_{AB}=17 Hz, H-21).

MS: 462, 464 (M⁺+1) 21-bromo;

MS: 418, 420 (M⁺+1) 21-chloro.

### EXAMPLE 3

This example relates to step c) of the process of the invention, from intermediate (III) to intermediate (IV), mixture 21-chloro/21-bromo-17α-hydroxypregn-4-en-3,20-dione:

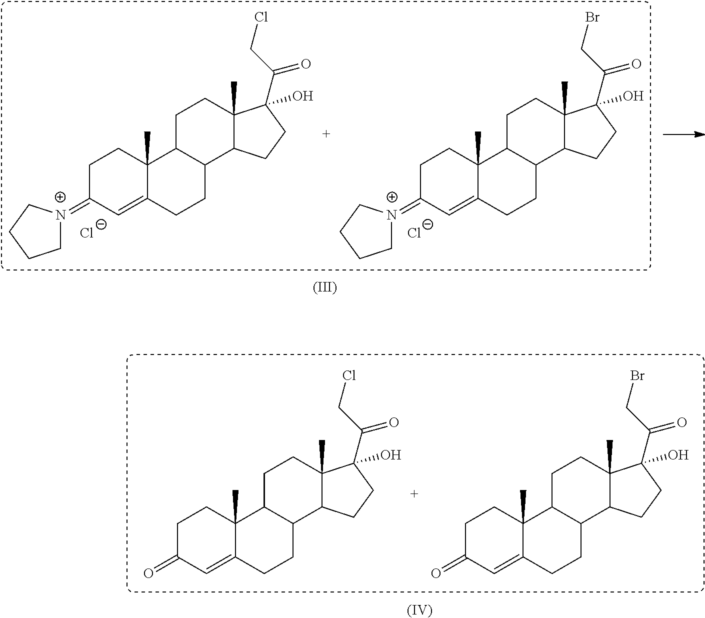

105.5 g of intermediate (III) obtained in the previous example are dissolved in 1582 ml of methanol; an aqueous solution of potassium bicarbonate (114.5 g of KHCO₃ in 458 g of water) is added, and the mixture is kept under stirring at 25° C. for about 5 hours; complete reaction is detected by TLC. 2000 ml of water are added, and the mixture is kept under stirring for 30 minutes. It is filtered through Buchner washing with 500 ml of water.

The product is dried in oven at 50° C. under vacuum for 16 h, obtaining 75.1 g of an off-white solid, which is recrystallized with 225 ml of a 1:1 (v/v), MEK-methanol mixture, obtaining 70.2 g of intermediate (IV) as an off-white solid.

Intermediate (IV) Analysis:

¹H-NMR, DMSO-d₆: 5.63 (1H, s, H-4); 5.56 (1H, s, OH-17); 4.58 (1H, part A of an AB system, J_{AB}=15 Hz, H-21); 4.35 (1H, part B of an AB system, J_{AB}=15 Hz, H-21); 2.60-2.53 (1H, m); 2.45-2.36 (2H, m); 2.26-2.13 (2H, m); 1.99-1.94 (1H, m); 1.84-1.18 (11H, m); 1.14 (3H, s, H-19); 1.05-0.94 (1H, m); 0.92-0.85 (1H, m); 0.56 (3H, s, H-18).

In the spectrum there are also the following peaks belonging to the 21-chloro-derivative: 5.54 (1H, s, OH-17); 4.77 (1H, part A of an AB system, J_{AB}=17 Hz, H-21); 4.46 (1H, part B of an AB system, J_{AB}=17 Hz, H-21).

MS: 409, 411 (M⁺+1) 21-bromo; 365, 367 (M⁺+1) 21-chloro.

### EXAMPLE 4

This example relates to step d) of the process of the invention, from intermediate (IV) to obtain compound (V), 21 -acetoxy -17α-hydroxypregn-4-en-3,20-dione:

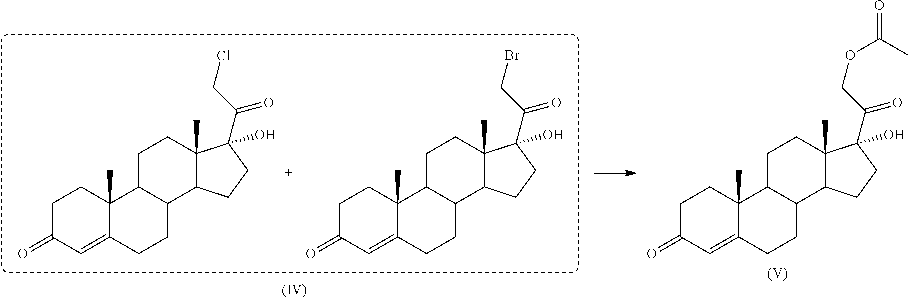

70 g of intermediate (IV) obtained in the previous example are suspended in 2100 ml of acetone, under a nitrogen flow. 190.8 ml of TEA and 39.2 ml of glacial acetic acid are added and heated to reflux (58° C.). A clear solution is never observed. After 5 h the reaction is practically complete. The solvent is removed by rotavapor, the residue is taken up with water (650 ml) and DCM (450 ml) and the layers are separated. The aqueous layer is re-extracted with DCM (100 ml) and the combined organic layers are washed with water (2×400 ml). The solvent is removed by rotavapor and 400 ml of MEK are added. The solvent is removed by rotavapor until a paste is obtained. The operation is repeated with additional 400 ml of MEK. 400 ml of MEK are added and the solvent is removed until a volume of about 350 ml of mixture is obtained. The mixture is cooled to 0° C. for 1 h and filtered through Buchner, washing with cold MEK (80 ml). The product is dried in oven at 45° C. under vacuum obtaining 59.1 g of compound (V) as a white solid.

Compound (V) Analysis:

¹H-NMR, CDCl₃: 5.73 (1H, s, H-4); 5.08 (1H, part A of an AB system, J_{AB}=17 Hz, H-21); 4.87 (1H, part B of an AB system, J_{AB}=17 Hz, H-21); 2.76-2.69 (1H, m); 2.72 (1H, s, OH-17); 2.48-2.26 (4H, m); 2.17 (3H, s, CO—CH₃); 2.07-2.01 (1H, m); 1.90-1.33 (11H, m); 1.19 (3H, s, H-19); 1.15-1.04 (1H, m); 1.01-0.94 (1H, m); 0.72 (3H, s, H-18).

MS: 389 (M⁺+1).

### EXAMPLE 5

This example relates to step e) of the process of the invention, from compound (V) to compound (VI), 21-(acetyloxy)-17-(1-oxopropoxy)-pregn-4-ene-3,20-dione, object of the invention:

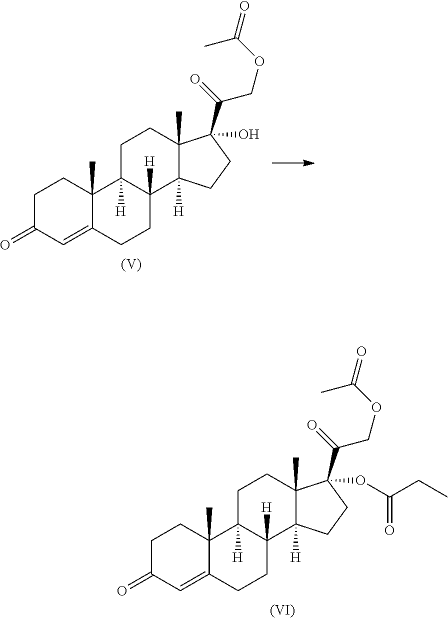

28.8 g of compound (V) obtained in the previous example are dissolved in 720 ml of DCM, under a nitrogen flow. 71.1 ml of propionic anhydride are added, and the mixture is cooled to −20° C. 7.3 ml of a 70% by weight HClO₄ aqueous solution are added, and an exothermic is observed from −20 to −15° C. The mixture is kept under stirring for 10 minutes at −20° C. The reaction is complete, and the reaction mixture is poured into 650 ml of NaHCO₃ saturated aqueous solution, keeping it under stirring for 30 min. The layers are separated, and the aqueous layer is re-extracted with 100 ml of DCM. The combined organic layers are washed with water (2×300 ml). DCM is removed by rotavapor under vacuum until a paste is obtained. 350 ml of heptane are added, and the solvent is removed until a paste is obtained. The operation is repeated with additional 350 ml of heptane. Finally, 350 ml of heptane are added, and the solvent is distilled until a residual volume of about 290 ml of mixture is obtained. The mixture is kept under stirring for 1 h at 25° C. and filtered through Buchner, washing with heptane. The product is dried in oven at 45° C. under vacuum obtaining 32.3 g of an off-white solid (compound (VI)).

Compound (VI) Analysis:

¹H-NMR, CDCl₃: 5.75 (1H, s, H-4); 4.89 (1H, part A of an AB system, J_{AB}=16 Hz, H-21); 4.63 (1H, part B of an AB system, J_{AB}=17 Hz, H-21); 2.88-2.81 (1H, m); 2.49-2.27 (6H, m); 2.17 (3H, s, CO—CH₃); 2.08-2.03 (1H, m); 1.95-1.60 (9H, m); 1.53-1.34 (2H, m); 1.20 (3H, s, H-19); 1.17-1.10 (1H, m); 1.16 (3H, t, J=7 Hz, CH₂—CH₃); 1.07-0.99 (1H, m); 0.76 (3H, s, H-18).

MS: 445 (M⁺+1).

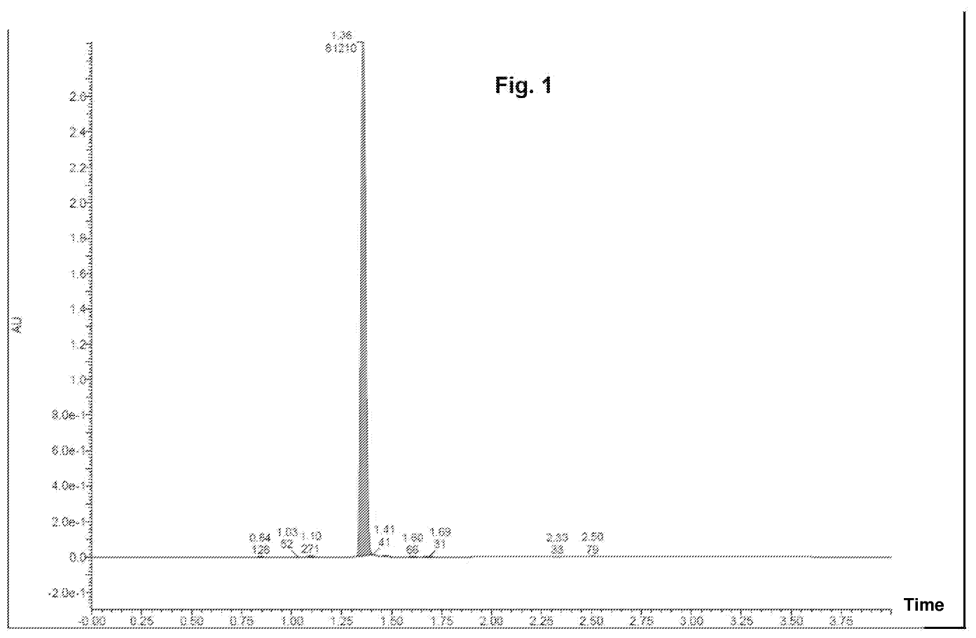

HPLC (purity): 99%, the chromatogram is shown in FIG. 1.

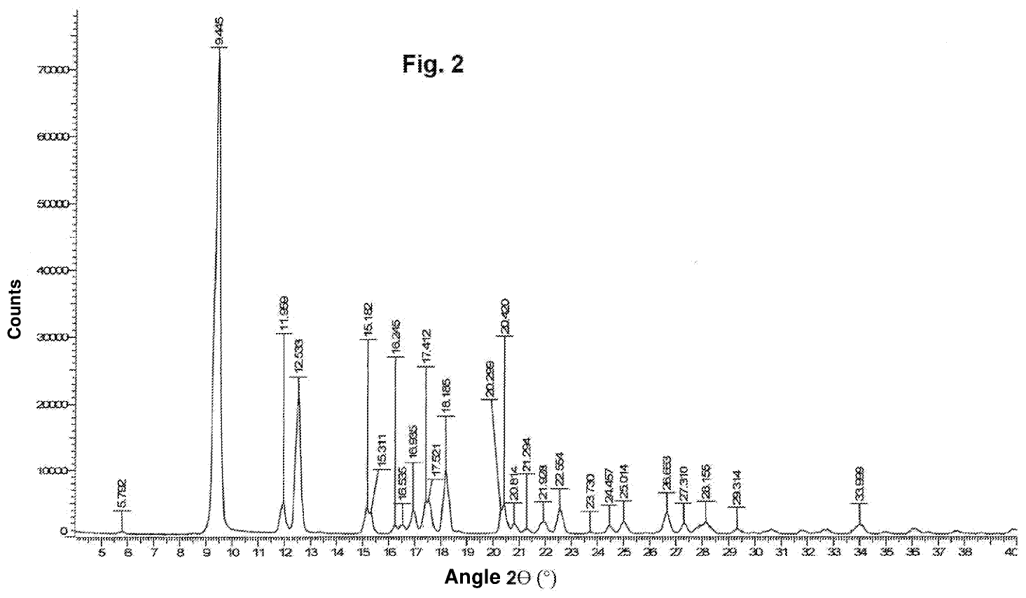

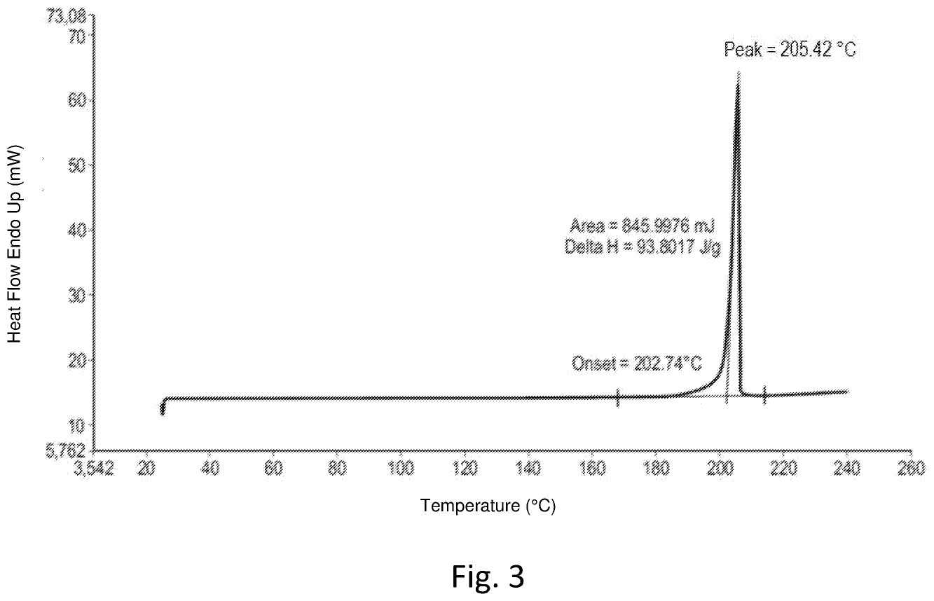

The sample is also subjected to DSC and XPRD analysis under the above test conditions; the results of the two tests are shown in FIGS. 2 and 3.

### EXAMPLE 6

This example relates to the hydrolysis of compound (VI) of the invention to Clascoterone using a supported enzyme in a flow reactor.

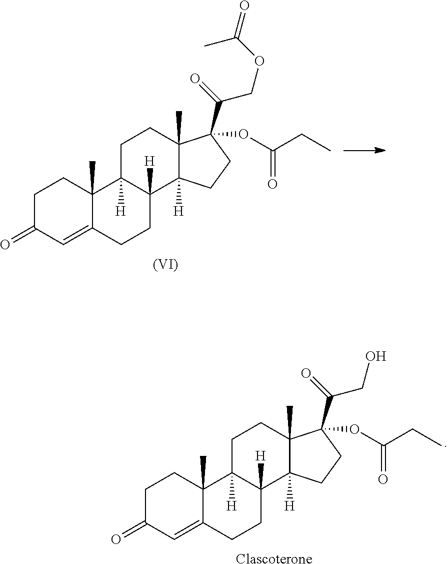

The process is carried out using a Vapourtec easy-Medchem E-series flow reactor in which the supplied tubular reactor is packed with 845 mg of Novozym® 435 (Candida Antarctica Lipase B supported on acrylic resin).

14.28 g of compound (VI) are dissolved in 1000 ml of toluene in a bottle designed for connecting to the flow reactor; 7.5 ml of n-butanol are added and stirred to dissolution. The solution is flowed through the tubular reactor packed with the enzyme, thermostated at 60° C., with a flow rate of 0.1 ml/min.

The progress of the conversion to Clascoterone is monitored by UPLC-MS analysis on the reaction solution sampling.

The enzyme efficiency, as it can be seen from the data shown in the following table, remains unchanged even after more than 100 hours of constant flow reaction.

| | | Clascoterone: UPLC |
|---|---|---|
| Control | Total flow time (h) | chromatogram % area |
| 1 | 1 | 95.66 |
| 2 | 9 | 95.55 |
| 3 | 22.5 | 96.34 |
| 4 | 33 | 96.19 |
| 5 | 47 | 97.00 |
| 6 | 57 | 96.63 |
| 7 | 71 | 97.28 |
| 8 | 81 | 96.33 |
| 9 | 105 | 96.98 |

### EXAMPLE 7

This example relates to the hydrolysis of compound (VI) to Clascoterone using a supported enzyme in a traditional closed reactor.

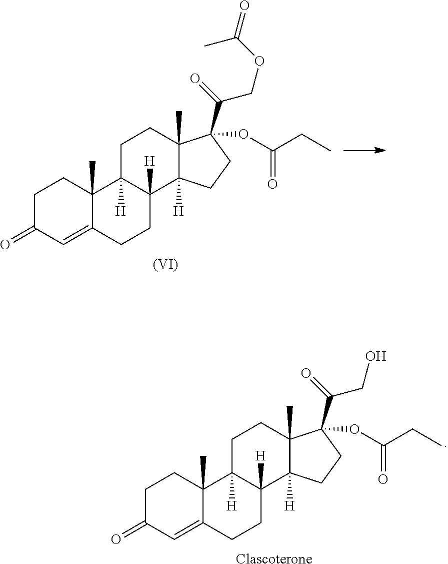

In a 100 ml glass reactor, 250 mg of 21-acetoxy-17α-propoxy-progesterone (VI) are dissolved in 17.5 ml of toluene, 250 mg of Novozym® 435 (Candida Antarctica Lipase B supported on acrylic resin), and finally 257 ill of n-butanol, are added. The mixture is stirred and brought to 60° C., monitoring the progress of the reaction over time by UPLC analysis.

After 14 hours and 30 minutes of stirring, the amount of residual compound (VI) and Clascoterone produced in the reaction mixture are of 0.75% and 96%, respectively, as measured by the area of the respective peaks in the UPLC chromatogram.

### EXAMPLE 8

This example relates to the enzymatic hydrolysis of compound (VI) of the invention to Clascoterone in comparison with the hydrolysis, in turn enzymatic, of the symmetric diester 17,21-bis(1-oxopropoxy)-pregn-4-ene-3,20-dione (VII) described in WO 2009/019138 operating in a conventional type reactor.

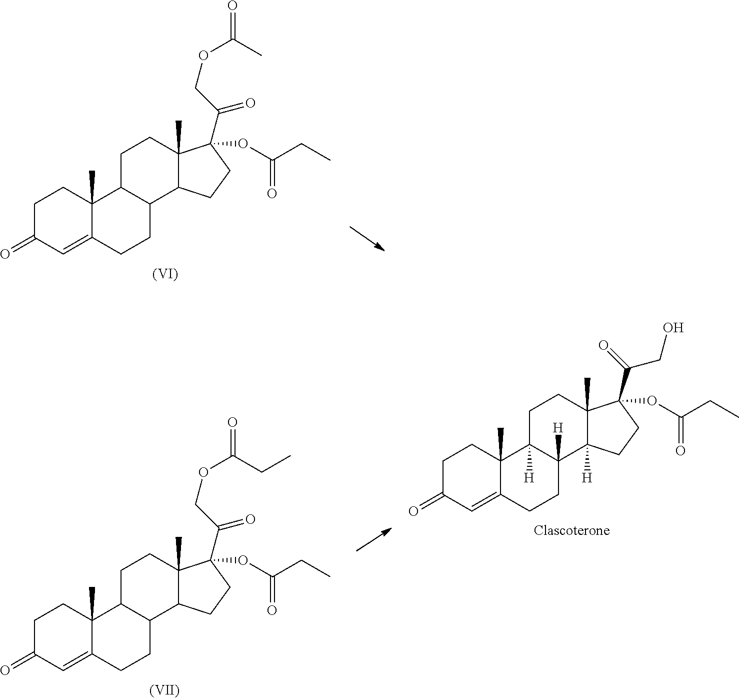

In a 50 ml glass flask, 250 mg of 21-acetoxy-17α-propoxy-progesterone (compound (VI), UPLC purity=99.7%) are dissolved in 17.5 ml of toluene; 250 mg of Novozym® 435 (Candida Antarctica Lipase B supported on acrylic resin), and finally 257 μl di n-butanol are added. The mixture is stirred and brought to 60° C., monitoring the reaction over time by UPLC analysis.

The experiment is repeated under identical conditions with 17,21-dipropoxy-17α-progesterone (compound (VII), UPLC purity =99.5%) except that, due to the higher molecular weight of compound (VII) compared to compound (VI), 258 mg of compound (VII) are used.

The progress of the conversion to Clascoterone is controlled by UPLC-MS analysis and the obtained results are reported in the following table (Clascoterone is denoted in the table as CLA). As it can be seen, the hydrolysis of compound (VI) proceeds faster than the hydrolysis of compound (VII).

| Reaction hours | 0 | 4 | 8 | 11.15 | 14.3 | 23.2 |
|---|---|---|---|---|---|---|
| (VI) | 99.7 | 24.6 | 5.34 | 1.75 | 0.75 | 0.47 |
| (VII) | 99.5 | 36.9 | 12.9 | 5.27 | 2.25 | 0.64 |
| CLA from (VI) | 0 | 74.3 | 90.2 | 93.4 | 96.0 | 96.5 |
| CLA from (VII) | 0 | 61.9 | 85 | 90.3 | 94.8 | 95.6 |

### EXAMPLE 9

This example relates to the obtainment of Clascoterone solvated with DMSO.

445 ml of solution obtained at the end of the reaction described in Example 6 are concentrated at 50° C. and under reduced pressure until 14.3 g of solution are obtained. 6.2 ml of dimethyl sulfoxide are then added, and evaporation is continued at 50° C. and reduced pressure until a solution is obtained in which the solvent is comprised of at least 99% of dimethyl sulfoxide (GC control). The solution is then placed under stirring at 20-25° C. for 16 hours obtaining the precipitation of a solid that, after filtration, is subjected wet to XPRD analysis. The obtained diffractogram is reproduced in FIG. 4; the list of the main diffractogram peaks, characterized by 2θ (±0.2°) angular positions and the relative intensities, is reported in the following table:

| 2θ (°) | Intensity (%) |
|---|---|
| 11.38 | 47.3 |
| 12.74 | 51.8 |
| 15.71 | 96.2 |
| 15.79 | 100.0 |
| 16.50 | 32.4 |
| 17.78 | 65.5 |
| 18.39 | 48.3 |
| 18.76 | 25.4 |
| 19.61 | 79.2 |
| 19.71 | 86.4 |
| 20.06 | 40.3 |

The wet solid (3.5 g) is then dissolved in 3.5 ml of dimethyl sulfoxide at 60° C. under stirring, then the solution is cooled to 25° C. over about 1 hour and left under stirring for 4 hours.

The precipitated solid, isolated by filtration, is dried under reduced pressure at 40° C. for 16 hours (2.6 g of white solid) and subjected to XPRD, DSC, FT-IR, ¹H-NMR (CDCl₃) analysis. The XPRD diffractogram on the dry product is identical to that obtained on the wet product (FIG. 4); the DSC thermogram and the FT-IR spectrum are reproduced in FIGS. 5 and 6 respectively (FIG. 5 shows the significant range, from 25 to 155° C., of the DSC thermogram). The NMR spectrum shows that the solid is a solvate of Clascoterone and dimethyl sulfoxide in a 1:1 molar proportion.

HPLC Purity: >99%.

### EXAMPLE 10

This example refers to the enzymatic hydrolysis of compound (VI) of the invention to Clascoterone in comparison with the hydrolysis, in turn enzymatic, of the symmetric diester 17,21-bis(1-oxopropoxy)-pregn-4-ene-3,20-dione, compound (VII), operating in a flow reactor by means of an enzyme supported on an inert material.

1.01 g of compound (VII) (purity 99.5%) are dissolved in 68.5 ml of toluene, 1.01 ml of n-butanol are added and the mixture is stirred to dissolution.

The solution thus obtained is passed through a tubular reactor previously packed with 1.068 g of Novozym® 435, operating at a flow of 0.134 ml/min, residence time of 19.1 min at 60° C. The instrument used is a Vapourtec easy-Medchem E-series.

Operations are carried out in a similar way with a solution of compound (VI) (purity 99.6%), again in toluene and n-butanol, under the same conditions.

The progress of the conversion to Clascoterone is controlled by UPLC-MS analysis by sampling the reaction solution.

The UPLC peak area ratio of unreacted compound (VII) to unreacted compound (VI) is 1.64.

### EXAMPLE 11

This example refers to the obtainment of Clascoterone solvated with DMSO via the metastable solvate from methanol.

632 ml of a solution obtained at the end of the reaction described in Example 6 are distilled at 50° C. under reduced pressure until a weight of 9 g is reached.

The solution is taken up with methanol and concentrated under reduced pressure and 50° C. for 3 times (using 26.4 ml of methanol for each dilution/concentration cycle) operating in such a way to always have a solution.

8.8 ml of methanol are added, and the solution is stirred first at 20-25° C. for 30 minutes, then at 4° C. for 6 hours.

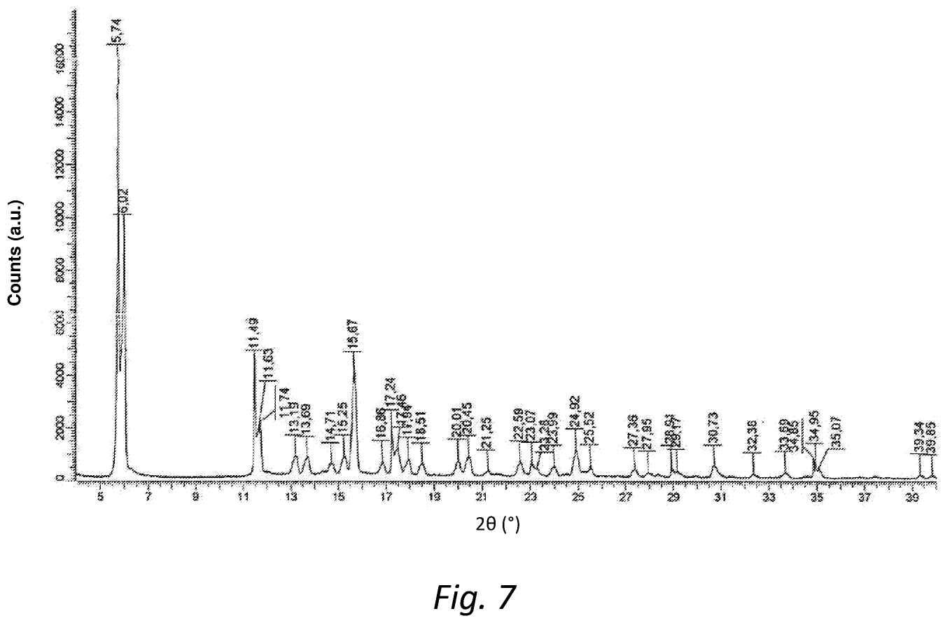

The suspension thus obtained is filtered to obtain a white solid, whose XPRD diffractogram (wet product) is immediately recorded highlighting a solid phase different from any known forms. The XPRD diffractogram of this compound is reproduced in FIG. 7; the list of the main peaks of the diffractogram, characterized by the 20 angular positions and relative intensities, is reported in the following table:

| 2θ (°) | Intensity (%) |
|---|---|
| 5.74 | 100.0 |
| 6.02 | 58.6 |
| 11.49 | 25.2 |
| 11.63 | 11.4 |
| 11.74 | 13.1 |
| 15.67 | 24.4 |
| 17.24 | 10.4 |

The wet solid is rapidly dissolved in 7 ml of dimethyl sulfoxide.

The solution thus obtained is distilled under reduced pressure at 50° C. to eliminate any methanol residue, then it is stirred at room temperature for 4 hours.

The suspension thus obtained is filtered and the solid is dried in an oven under vacuum at 40° C. for 16 hours.

2.3 g of white solid are obtained, whose XPRD diffractogram corresponds to that of Clascoterone solvated with DMSO shown in FIG. 4.

### EXAMPLE 12

This example relates to obtaining Clascoterone solvated with DMSO.

3100 ml of reaction solution obtained in the same way as described in Example 6, and containing about 40 g of Clascoterone, are concentrated at 50° C. and reduced pressure until 144.2 g of solution are obtained. 40 ml of DMSO are added and concentration of the solution is continued at 50° C. and reduced pressure up to a final weight of 84.9 g. The solution is heated to 65° C., cooled to 20° C. in about 1 h, and left under stirring for 22 h (the precipitation of a solid is observed). After filtration, the wet product is dried under reduced pressure at 40° C. for 20 h to obtain 34.3 g of Clascoterone solvated with DMSO (white solid, UPLC purity=99.19%).

Clascoterone solvated with DMSO (34.3 g) is further purified by recrystallization. The solvate is mixed with 23.4 ml of DMSO. The suspension is heated to 65° C., kept under stirring for 10 min, then cooled to 20° C. in 1 hour, and left under stirring at 20° C. for 22 h. The suspension is filtered, and the wet solid is dried in oven at 40° C. and under reduced pressure for 20 h to obtain Clascoterone solvated with DMSO as a white solid having UPLC purity=99.70%.

### EXAMPLE 13

This example refers to obtaining Clascoterone solvated with DMSO by triggering with a crystalline seed.

3350 ml of reaction solution obtained in the same way as described in Example 6, containing 43 g of Clascoterone, are concentrated at 50° C. under reduced pressure until 93.2 g of solution are obtained. 43 ml of DMSO are added and the distillation is continued under reduced pressure to a final weight of 92.5 g. The solution is heated to 65° C., stirred for 10 min, then cooled to 50° C. in about 15 min. 0.23 g of Clascoterone solvated with DMSO obtained by the procedure described in Example 12 are added, and left under stirring for 10 min. The suspension is cooled to 20° C. in 1 h, then it is left under stirring at 20° C. for 18 h. The obtained solid is filtered, then dried in oven at 40° C. and under reduced pressure for 20 h, obtaining 43.4 g of Clascoterone solvated with DMSO (white solid, UPLC purity=99.41%)

Also in this case, as for Example 12, the obtained Clascoterone solvated with DMSO can be recrystallized from DMSO until the desired level of purity is obtained.

1-14. (canceled)

15. A process for the synthesis of 21-(acetyloxy)-17-(1-oxopropoxy)-pregn-4-ene-3,20-dione, compound of formula (VI): 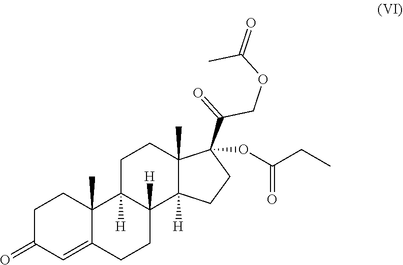
comprising the following steps:
a) reaction of 17α-hydroxy-progesterone (I) with pyrrolidine to give compound (II), 17-hydroxy-3-(1-pyrrolidinyl)pregna-3,5-dien-20-one: 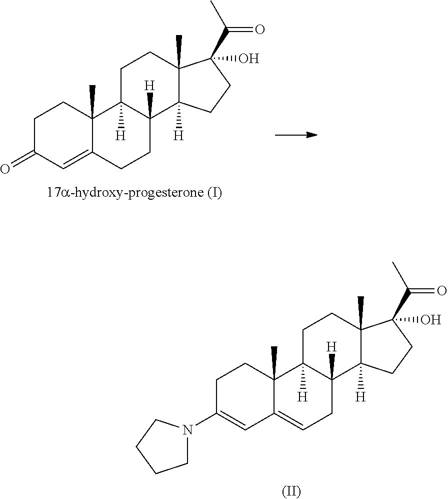
b) reaction of compound (II) first with hydrochloric acid then with bromine to give intermediate (III), a mixture of (21-chloro/21-bromo)-17α-hydroxy-3-(1-pyrrolidinium-1-yliden)-pregn-4-en-20-one chloride: 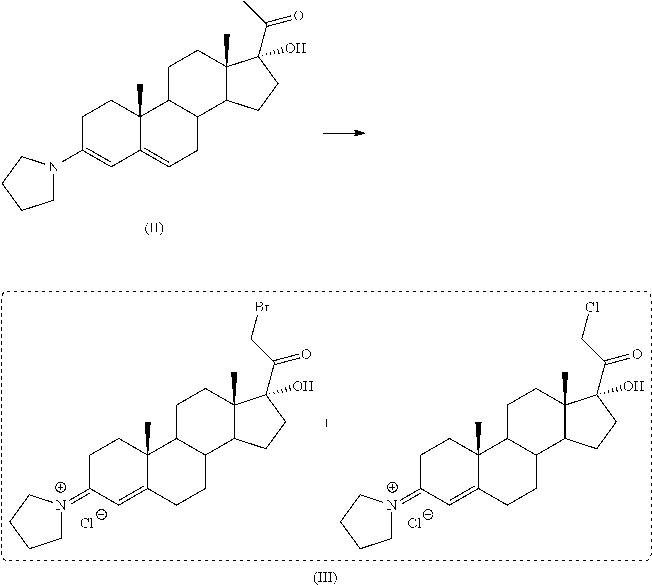
c) basic hydrolysis of intermediate (III) to obtain intermediate (IV), the corresponding mixture of (21-chloro/21-bromo)-17α-hydroxypregn-4-en-3,20-dione: 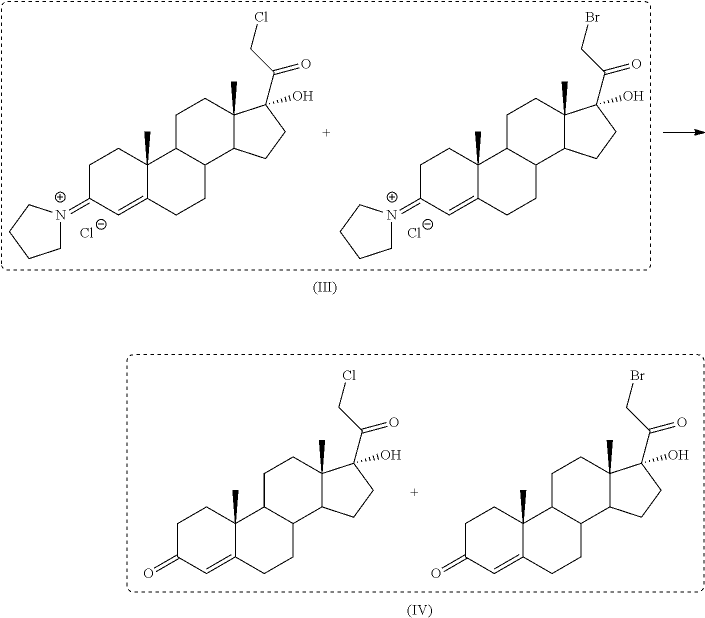
d) reaction of intermediate (IV) with acetic acid to obtain compound (V), 21-acetoxy-17α-hydroxypregn-4-en-3,20-dione (V): 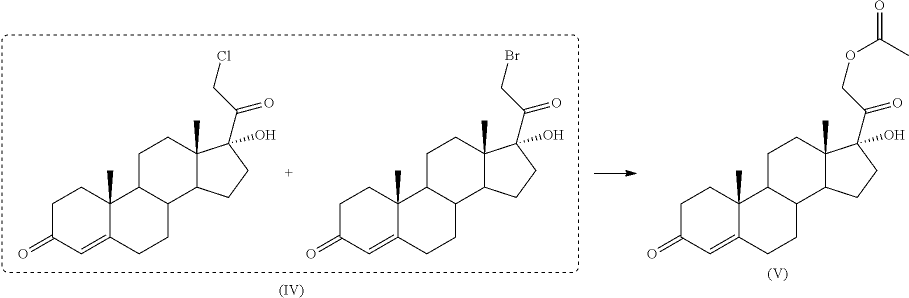
e) reaction of compound (V) with perchloric acid and propionic anhydride to obtain compound (VI), 21-(acetyloxy)-17-(1-oxopropoxy)-pregn-4-ene-3,20-dione:

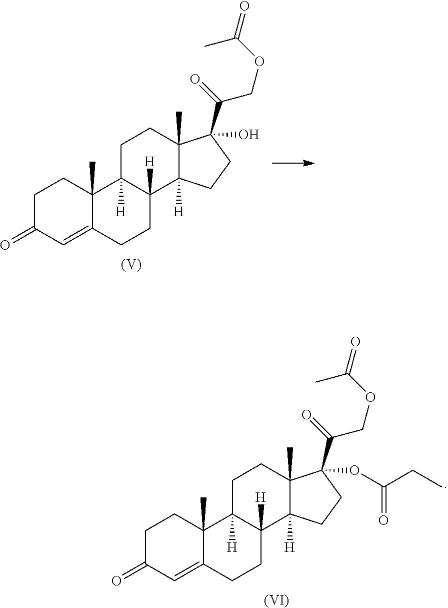

16. The process according to claim 15, further comprising a step f) of selective hydrolysis of the compound 21-(acetyloxy)-17-(1-oxopropoxy)-pregn-4-ene-3,20-dione, compound of formula (VI), to give the compound 21-hydroxy-17-(1-oxopropoxy)-pregn-4-ene-3,20-dione (Clascoterone).

17. The process according to claim 16, wherein step f) is carried out by acid hydrolysis.

18. The process according to claim 16, wherein step f) is carried out by enzymatic hydrolysis.

19. The process according to claim 18, wherein said enzymatic hydrolysis is carried out using a supported lipase as a reactant.

20. The process according to claim 18, wherein operations are carried out in a flow reactor.

21. The process according to claim 20, wherein operations are carried out in the presence of toluene and an alcohol.

22. The process according to claim 21, wherein said alcohol is n-butanol.

23. The compound 21-(acetyloxy)-17-(1-oxopropoxy)-pregn-4-ene-3,20-dione (VI):

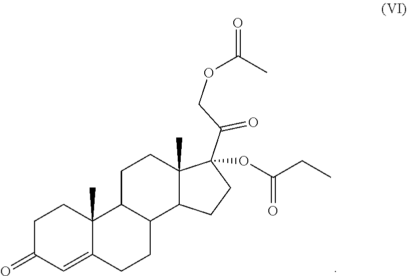 [SMILES: CCC(=O)O[C@]1(C(=O)COC(C)=O)CCC2C3CCC4=CC(=O)CC[C@]4(C)C3CC[C@@]21C]

24. A solvate of Clascoterone with dimethyl sulfoxide.

25. The solvate according to claim 24, wherein the Clascoterone:dimethyl sulfoxide molar ratio is 1:1.

26. A pharmaceutical composition comprising the solvate of claim 24 and pharmaceutically acceptable excipients.

27. A pharmaceutical composition comprising the solvate of claim 25 and pharmaceutically acceptable excipients.
